(19) United States
(12) Patent Application Publication
(10) Pub. No.: US 2025/0262183 A1
(43) Pub. Date: Aug. 21, 2025

(54) Antibiotic Topical Composition for Absorption into Systemic Bloodstream

(71) Applicant: Harvey Tenenbaum, Toronto (CA)

(72) Inventor: Harvey Tenenbaum, Toronto (CA)

(21) Appl. No.: 18/581,002

(22) Filed: Feb. 19, 2024

(51) Int. Cl. A61K 31/353; A61K 9/00; A61K 31/43; A61K 31/496; A61K 31/5383; A61K 31/545; A61K 31/546; A61K 31/7048

(52) U.S. Cl. CPC A61K 31/353; A61K 9/0014; A61K 31/43; A61K 31/496; A61K 31/5383; A61K 31/545; A61K 31/546; A61K 31/7048

(57) ABSTRACT

A transdermal antibiotic composition and method of administration is provided. The antibiotic topical includes a lotion containing an antibiotic, a suspension agent, and a transdermal agent for dermal penetration and absorption. This composition allows for the administration of antibiotics through the skin, bypassing the gastrointestinal system, and avoiding adverse reactions associated with oral ingestion or injections. The suspension agent includes polyphenols, wherein the transdermal agent is able to provide for dermal penetration and the absorption of antibiotics in the bloodstream.

### BACKGROUND OF THE INVENTION

The invention relates to the field of pharmaceutical formulations and medical devices, specifically addressing the systemic administration of antibiotics. It encompasses transdermal delivery systems for antibiotics, aiming to provide a safe, effective, and convenient alternative to traditional methods of antibiotic administration.

Antibiotics have been a cornerstone in the treatment of bacterial infections for decades, commonly administered orally or through injections. However, these conventional methods of antibiotic administration are not without limitations and drawbacks including adverse effects on the gastrointestinal system and risks associated with systemic delivery methods. Some adverse effects on the gastrointestinal system including nausea, diarrhea, and disruption of the natural gut microbiota. There are also questions of bioavailability from oral administration of antibiotics indicate a clear knowledge gap regarding the bioavailability of orally administered antibiotics in non-ICU patients during the initial phase of a systemic infection.

In addition to the inherent risks associated with traditional antibiotic treatments, there are limitations in the efficacy of systemic administration methods. Oral antibiotics may not effectively target specific areas requiring treatment, leading to suboptimal therapeutic outcomes. Moreover, systemic absorption of antibiotics can be variable and unpredictable, resulting in fluctuations in drug concentrations in the bloodstream and potential under-dosing or overdosing.

Advances in independent research techniques have led to an increased understanding of the gut microbiota and the role it plays in health and disease. The intestine is populated by a complex microbial community that is organized around a network of metabolic interdependencies. The gut microbiota is vital for normal development and functioning of the human body, especially for the priming and maturation of the adaptive immune system.

The intestinal microbiota plays beneficial roles in many physiological processes of the host. It extracts energy and nutrition from food, protects against enteropathogens, and supports development and maintenance of the host immune system. The biodiversity of the intestinal microbiota among individuals implies that it sustains a homeostatic equilibrium state against a decrease in its composition and function. The particular interrelationship between the intestinal microbiota and the host is a product of long-term coexistence and evolution. Dysbiosis, a disruption of microbial composition by various stresses, has been implicated in inflammatory bowel disease (IBD), colon cancer, obesity, asthma, and other diseases. Antibiotics trigger the proliferation of intestinal pathogenic bacteria due to the ability of infectious bacteria to effectively exploit the disorder that arises when the intestinal microbiota has collapsed.

Antibiotic use can have several negative effects on the gut microbiota, including reduced species diversity, altered metabolic activity, and the selection of antibiotic-resistant organisms, which in turn can lead to antibiotic-associated diarrhea and recurrent Clostridioides difficile infections. There is also evidence that early childhood exposure to antibiotics can lead to several gastrointestinal, immunologic, and neurocognitive conditions. The increase in the use of antibiotics in recent years suggests that these problems are likely to become more acute or more prevalent in the future. Continued research into the structure and function of the gut microbiota is required to address this challenge.

Transdermal administration of antibiotics offers several potential advantages over oral ingestion and injections. By bypassing the gastrointestinal system, transdermal delivery minimizes the risk of gastrointestinal side effects and allows for targeted delivery to the site of infection. Furthermore, transdermal absorption provides a more controlled and sustained release of antibiotics, maintaining consistent therapeutic levels in the bloodstream and improving treatment efficacy.

Despite the potential benefits of transdermal antibiotic administration, there are challenges associated with achieving effective dermal penetration and absorption of antibiotics. The stratum corneum, the outermost layer of the skin, serves as a barrier to drug penetration, limiting the absorption of topical medications. Additionally, factors such as the molecular size and lipophilicity of antibiotics can influence their ability to penetrate the skin barrier and reach therapeutic concentrations in the systemic circulation.

In view of these developments, there exists a need for a transdermal antibiotic topical that provides for the administration of antibiotics to the bloodstream, thereby circumventing the gastrointestinal system, and avoiding adverse reactions associated with oral ingestion or injections. The present invention addresses these challenges by providing a transdermal antibiotic topical solution formulated with specific antibiotics, polyphenols, and transdermal agents. This formulation enhances dermal penetration and absorption of antibiotics, ensuring systemic delivery while minimizing adverse reactions and optimizing therapeutic outcomes. By harnessing the synergistic effects of polyphenols and utilizing transdermal agents, the composition offers a safe, effective, and convenient alternative to traditional methods of antibiotic administration.

In light of the devices disclosed in the known art, it is submitted that the present invention substantially diverges in design elements and methods from the known art and consequently it is clear that there is a need in the art for an improvement in antibiotic topical compositions and methods of administration thereof. In this regard the instant invention substantially fulfills these needs.

### SUMMARY OF THE INVENTION

In view of the foregoing disadvantages inherent in the known types of antibiotic topical composition and method of application. The antibiotic topical composition comprises an antibiotic, a suspension agent, and a transdermal agent specifically formulated for dermal penetration and absorption. This composition facilitates the systemic administration of antibiotics via transdermal application, effectively circumventing the gastrointestinal tract and mitigating potential adverse reactions commonly associated with oral ingestion or parenteral injections. The suspension agent incorporates specific polyphenolic compounds which synergistically enhances the therapeutic efficacy of the formulation. Concurrently, the transdermal agent provides for dermal penetration. The composition ensures that the antibiotics are bioavailable while minimizing systemic side effects, thereby representing a promising advancement in antibiotic delivery methodologies.

It is an objective of the present invention to provide an embodiment of the antibiotic topical composition to enhance the effectiveness of antibiotic treatment. This is achieved by synergistically combining antibiotics with polyphenols, which have been demonstrated to directly kill bacteria, activate antibiotics, and attenuate bacterial pathogenicity.

It is an objective of the present invention to provide an embodiment of the antibiotic topical composition to minimize adverse reactions commonly associated with traditional routes of antibiotic administration. By delivering antibiotics transdermally, the composition bypasses the gastrointestinal system, reducing the likelihood of systemic side effects. This approach aims to enhance patient safety and treatment adherence by minimizing adverse reactions and discomfort associated with oral ingestion or parenteral injections.

It is an objective of the present invention to provide an embodiment of the antibiotic topical composition to ensure safe and effective systemic antibiotic delivery through the skin. Additionally, it aims to offer customizable solutions for antibiotic delivery, tailored to individual patient needs and preferences. The concentration of antibiotics and the dosage is modifiable for each individual and diagnosis.

It is also another objective of the present invention to provide an embodiment of the antibiotic topical composition that optimizes the bioavailability of antibiotics. This is achieved by formulating a transdermal delivery system that facilitates efficient dermal penetration and absorption into the bloodstream. By ensuring optimal bioavailability, the composition aims to achieve therapeutic levels of antibiotics, thereby maximizing the effectiveness of antibiotic treatment

It is an objective of the present invention to provide an embodiment of the antibiotic topical composition that is formulated as a lotion. The topical formulation can easily be applied to the skin which promotes patient compliance and treatment adherence. This delivery recognizes the importance of convenience and patient comfort in ensuring the successful implementation of antibiotic treatment regimens.

It is therefore an object of the present invention to provide a new and improved antibiotic topical composition that has all of the advantages of the known art and none of the disadvantages.

Other objects, features and advantages of the present invention will become apparent from the following detailed description taken in conjunction with the accompanying drawings.

### DETAILED DESCRIPTION OF THE INVENTION

For the purpose of presenting a brief and clear description of the present invention, the embodiment discussed will be used for topical application of the composition to deliver antibiotics as part of a therapeutic treatment, demonstrating its efficacy in addressing bacterial infections and enhancing patient outcomes. The described features, structures, or characteristics may be combined in any suitable manner in one or more embodiments. In the following description, numerous specific details are provided to give a thorough understanding of embodiments.

Reference will now be made in detail to the exemplary embodiment(s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment,” “first embodiment”, “second embodiment”, or “third embodiment” does not necessarily refer to the same embodiment.

The antibiotic topical composition provides for the transdermal administration of antibiotics to a patient and into the systemic bloodstream. The antibiotic topical composition penetrates the skin to deliver the antibiotic to the bloodstream of the user, while bypassing the gastrointestinal tract. The antibiotic topical composition comprises a transdermal agent, an antibiotic, and a suspension agent incorporating polyphenols. Transdermal agents, acting as chemical penetration enhancers (CPEs) or sorption promoters, facilitate the penetration of the antibiotic through the stratum corneum into the bloodstream. The suspension agent, containing polyphenols, synergistically interacts with antibiotics to combat bacterial infections by directly killing bacteria, activating antibiotic effects, and attenuating bacterial pathogenicity.

In this context, “systemic bloodstream” refers to the circulatory system of the body, which includes the heart, blood vessels, and blood. When the antibiotic topical composition is applied to the skin, the transdermal agents facilitate the penetration of antibiotics through the skin's layers and into the systemic bloodstream. Once absorbed into the systemic bloodstream, the antibiotics are transported throughout the body, reaching various tissues and organs to exert their therapeutic effects against bacterial infections.

In one embodiment, the synergy between polyphenols and antibiotics plays a crucial role in enhancing the antibacterial efficacy of the topical composition. Polyphenols, particularly flavonoids, exhibit antibacterial effects through multiple mechanisms. Firstly, they have been shown to directly kill bacteria, disrupting their cellular structures and inhibiting their growth. Secondly, polyphenols can synergistically activate antibiotics, enhancing their effectiveness against bacterial pathogens. This synergistic activation can lead to increased potency and efficacy of antibiotic treatments. Thirdly, polyphenols have the ability to attenuate bacterial pathogenicity, reducing the virulence and harmful effects of bacterial infections. When combined with antibiotics in the topical composition, polyphenols act synergistically to enhance the overall antibacterial activity. By directly killing bacteria, activating antibiotics, and attenuating bacterial pathogenicity, polyphenols amplify the therapeutic effects of antibiotics. This synergistic action ensures a more potent and effective treatment against bacterial infections. Moreover, the transdermal agents in the composition facilitate the bioavailability of both antibiotics and polyphenols without the need for oral ingestion or injections. By enabling efficient dermal penetration and absorption, these transdermal agents ensure that the therapeutic components reach the systemic circulation, where they can exert their antibacterial effects. Overall, the combination of natural polyphenols, antibiotics, and transdermal agents in the topical composition creates a synergistic effect that enhances antibacterial activity, improves bioavailability, and provides an effective alternative to traditional routes of administration.

Among the chemicals that increase skin permeability are those known as chemical penetration enhancers (CPEs) or sorption promoters. These include terpenes, terpenoids, sulfoxides, laurocapram (Azone), pyrrolidones, fatty acids, fatty alcohols, alcohols containing glycols, urea, and surfactants.

In some embodiments, the CPEs include the following groups of chemicals: alcohols, polyols, lactams and their analogues (azepane, azone, caprolactam, morpholine, piperazine, piperidine, piperidone, pyrrolidine, pyrrolidone, and succinimide), esters and ethers, fatty acids, terpenes and steroids, and miscellaneous additives such as amino acids, aliphatic compounds, aromatic compounds, and inorganic compounds.

In one embodiment, the transdermal formulation is based on natural substances that offer high potential for safe use and low risk of skin irritation. These ingredients, derived from natural sources, are biodegradable, readily available, and widely accepted by patients. In one embodiment, the natural substances include sterols, ceramides, fatty acids (such as oleic acid), and urea, wherein these ingredients are permeation enhancers found in the stratum corneum. Additionally, fatty acids and terpenes, predominantly sourced from plants, serve as natural enhancers. Plant oils, rich sources of fatty acids, possess skin barrier restoration, regenerative, antioxidant, and anti-inflammatory properties. While plant oils are generally considered safe for topical use, their effects on the skin may vary depending on factors such as the ratio of oleic to linoleic acids. The use of natural substances that facilitate API (Active Pharmaceutical Ingredient) permeation through the stratum corneum are used in this embodiment. API penetration is most effective when utilizing a compound formulation containing a mixture of permeation enhancers that interact with both lipids and proteins of the stratum corneum, creating transport pathways for both hydrophobic and hydrophilic APIs. This approach minimizes the risk of skin irritation and offers effective use of polar and nonpolar APIs, contributing to the advancement of transdermal drug delivery systems.

In the antibiotic topical composition, various classes of antibiotics are incorporated to provide a broad spectrum of antimicrobial activity against pathogenic bacteria. These antibiotics include macrolides, such as clarithromycin, which inhibit bacterial protein synthesis; cephalosporins, such as cefdinir and cefpodoxime, which disrupt bacterial cell wall synthesis; fluoroquinolones, such as ciprofloxacin and levofloxacin, which interfere with bacterial DNA replication and transcription, and penicillins, such as amoxicillin and ampicillin, which also disrupt bacterial cell wall synthesis. By incorporating multiple classes of antibiotics, the composition ensures efficacy against a wide range of bacterial strains, thereby enhancing the effectiveness of the topical treatment for various bacterial infections. In one embodiment, the antibiotic of the composition is selected from the group consisting of: macrolides, cephalosporins, fluoroquinolones, and penicillins.

The antibiotic topical composition is prepared through a systematic process involving several key steps. Initially, all ingredients, including the transdermal agent, the selected antibiotics, the suspension agent containing polyphenols, and any additional inert components, are gathered. These ingredients are measured and assembled in preparation for formulation. Once gathered, the suspension agent is prepared by blending its components to ensure proper dispersion of polyphenols within the suspension. This ensures that the desired viscosity and consistency of the final composition. Adjustments may be made as necessary to optimize the formulation. Subsequently, the transdermal agent is combined with the suspension, with careful attention paid to ensuring thorough mixing to achieve homogeneity. The incorporation of the transdermal agent facilitates the efficient delivery of the antibiotic through the skin barrier, enhancing the overall efficacy of the composition. Following the incorporation of the transdermal agent, the selected antibiotics are added to the suspension-transdermal agent mixture. Thorough mixing is performed to ensure the antibiotics are evenly dispersed within the formulation. In one embodiment, the mixture is continually mixed and monitored to ensure proper blending of all ingredients. Vigorous stirring or shaking is employed to achieve a uniform and homogenous composition. This mixing process provides for the stability and efficacy of the final composition. Upon completion of the formulation process, the antibiotic topical composition is ready for use. Instructions for use are provided, detailing the application method, dosage, and frequency.

In one embodiment, antibiotic topical composition includes polar and nonpolar elements, where in the ration of polar and nonpolar elements does not exceed 1:1. In the formulation of the antibiotic topical composition, the balance between polar and nonpolar elements ensured the desired characteristics of the composition. Polar elements, such as water and certain solvents, possess an uneven distribution of charge and exhibit strong intermolecular attractions. Nonpolar elements, such as oils and hydrocarbons, have a symmetrical distribution of charge and lack significant intermolecular attractions, making them effective solvents for nonpolar substances like lipids and hydrophobic compounds. In one embodiment, maintaining a balanced ratio of polar and nonpolar elements, where the ratio does not exceed 1:1, is provided for preserving the composition's lotion-like consistency or suspended state. This equilibrium ensures that the composition retains its desired physical properties, such as viscosity and stability, necessary for topical application. An excessive imbalance in favor of polar or nonpolar elements can lead to phase separation or alteration of the composition's texture. The composition provides for dermal penetration and absorption of antibiotics while providing a comfortable and user-friendly experience for patients.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An antibiotic topic composition for absorption into systemic bloodstream through skin, comprising:
a transdermal agent configured for dermal penetration and absorption through the stratum corneum into a bloodstream;
an antibiotic;
a suspension agent adapted to suspend the transdermal agent and the antibiotic;
wherein the suspension agent includes a polyphenol.

2. The antibiotic topical composition of claim 1, wherein the polyphenol is configured to activate the antibiotic, and the antibiotic and polyphenol serve to synergistically kill bacteria and attenuate bacterial pathogenicity.

3. The antibiotic topical composition of claim 1, wherein the transdermal agent is configured for dermal penetration and absorption, wherein the transdermal agent synergistically enhances the bioavailability of antibiotics by facilitating their efficient absorption into the bloodstream.

4. The antibiotic topical composition of claim 1, wherein the antibiotics is selected from the group consisting of: macrolides, cephalosporins, fluoroquinolones, and penicillins.

5. The antibiotic topical composition of claim 1, wherein the transdermal agent are chemical penetration enhancers (CPEs) or sorption promoters.

6. The antibiotic topical composition of claim 5, wherein the CPEs or sorption promoters is selected from the group consisting of: terpenes, terpenoids, sulfoxides, laurocapram (Azone), pyrrolidones, fatty acids, fatty alcohols, alcohols containing glycols, urea, and surfactants.

7. The antibiotic topical composition of claim 1, wherein the transdermal agent comprises less than 51% of the topical composition.

8. The antibiotic topical composition of claim 1, wherein the antibiotic topical composition includes polar and nonpolar elements, where in the ration of polar and nonpolar elements does not exceed 1:1.

9. The antibiotic topical composition of claim 1, wherein an amount of polyphenols of the suspension agent is less than 60% of a total volume of the antibiotic topical composition.

10. The antibiotic topical composition of claim 1, wherein the polyphenol directly contributes to the antibacterial efficacy of the composition by effectively killing bacteria and attenuating bacterial pathogenicity.

11. The antibiotic topical composition of claim 1, wherein the transdermal agent is configured for sustained release of antibiotics, wherein the transdermal agent prolongs the therapeutic effects of antibiotics by maintaining therapeutic concentrations in the bloodstream over an exposure period.

12. The antibiotic topical composition of claim 1, wherein the suspension agent is adapted to provide uniform dispersion of the polyphenol and antibiotic.
